(19) United States
(12) Patent Application Publication
(10) Pub. No.: US 2014/0354949 A1
(43) Pub. Date: Dec. 4, 2014

(54) INTERACTIVE PLATFORM FOR HEALTH ASSESSMENT

(71) Applicants: Sherwyne R. Bakar, Palo Alto, CA (US); Julia Lehman, San Francisco, CA (US); Patrick Doyle, Hillsborough, CA (US)

(72) Inventors: Sherwyne R. Bakar, Palo Alto, CA (US); Julia Lehman, San Francisco, CA (US); Patrick Doyle, Hillsborough, CA (US)

(21) Appl. No.: 14/292,116

(22) Filed: May 30, 2014

Related U.S. Application Data
(60) Provisional application No. 61/829,888, filed on May 31, 2013.
(60) Provisional application No. 61/831,467, filed on Jun. 5, 2013.

(51) Int. Cl. A61B 3/18; A61B 3/14; A61B 3/032

(52) U.S. Cl. CPC A61B 3/18; A61B 3/032; A61B 3/14; A61B 3/145
USPC 351/206; 351/223; 351/246

(57) ABSTRACT

Methods and systems are disclosed for performing an assessment of vision-related health conditions for a user. A questionnaire regarding a user's identity and health history is displayed on a display mounted to a station. At least one interactive test image for assessing visual acuity is displayed on the display mounted to a station. An assessment is performed of vision-related health conditions for the user based on the user's response to the at least one interactive test image. At least one professional services and one marketing option is displayed. Instructions to the user for obtaining a copy of the assessment of vision-related health conditions for the user are displayed.

### RELATED APPLICATIONS

This application claims priority to the co-pending provisional patent application Ser. No. 61/829,888, Attorney Docket Number JLPD-001.PRO, entitled “SYSTEM AND METHOD FOR VISION HEALTH TESTING,” with filing date May 31, 2013, which is herein incorporated by reference in its entirety.

This application also claims priority to the co-pending provisional patent application Ser. No. 61/831,467, Attorney Docket Number JLPD-002.PRO, entitled “INTERACTIVE KIOSK PLATFORM FOR HEALTH ASSESSMENT,” with filing date Jun. 5, 2013, which is herein incorporated by reference in its entirety.

### BACKGROUND

Many diseases are related to the eye and could be detected with regular eye testing. In some cases eye diseases are asymptomatic. Early detection of an eye disease may be a key to successful treatment. For these reasons, health care professionals often recommend periodic and thorough eye examinations as part of routine primary care. However, eye testing or examinations take time and typically require a health care professional such as an ophthalmologist or optometrist, which leads to increased costs. Therefore many people do not seek out regular eye examination.

### BRIEF DESCRIPTION OF THE DRAWINGS

The accompanying drawings, which are incorporated in and form a part of this application, illustrate and serve to explain the principles of embodiments in conjunction with the description. Unless noted, the drawings referred to this description should be understood as not being drawn to scale.

FIG. 1 is a block diagram of a station for performing an assessment of vision-related health conditions for a user in accordance with embodiments of the present technology.

FIG. 2 is a block diagram of a station for performing an assessment of vision-related health conditions for a user in accordance with embodiments of the present technology.

FIG. 3 is a block diagram of an example computer system upon which embodiments of the present technology may be implemented.

FIG. 4 is block diagram of an image used for performing an assessment of vision-related health conditions for a user in accordance with embodiments of the present technology.

FIG. 5 is block diagram of an image used for performing an assessment of vision-related health conditions for a user in accordance with embodiments of the present technology.

FIG. 6 is block diagram of an image used for performing an assessment of vision-related health conditions for a user in accordance with embodiments of the present technology.

FIG. 7 is block diagram of an image used for performing an assessment of vision-related health conditions for a user in accordance with embodiments of the present technology.

FIG. 8 is block diagram of an interface used for performing an assessment of vision-related health conditions for a user in accordance with embodiments of the present technology.

FIG. 9 is a flowchart of a method for performing an assessment of vision-related health conditions for a user in accordance with embodiments of the present technology.

FIG. 10 is a flowchart of a method for performing an assessment of vision-related health conditions for a user in accordance with embodiments of the present technology.

FIG. 11 is a flowchart of a method for assessing optical fatigue for a user in accordance with embodiments of the present technology.

FIG. 12 is a flowchart of a method for presenting healthcare options to a user of a medical testing device in accordance with embodiments of the present technology.

### DESCRIPTION OF EMBODIMENT(S)

Reference will now be made in detail to various embodiments of the present technology, examples of which are illustrated in the accompanying drawings. While the present technology will be described in conjunction with these embodiments, it will be understood that they are not intended to limit the present technology to these embodiments. On the contrary, the present technology is intended to cover alternatives, modifications and equivalents, which may be included within the spirit and scope of the present technology as defined by the appended claims. Furthermore, in the following description of the present technology, numerous specific details are set forth in order to provide a thorough understanding of the present technology. In other instances, well-known methods, procedures, components, and circuits have not been described in detail as not to unnecessarily obscure aspects of the present technology.

Unless specifically stated otherwise as apparent from the following discussions, it is appreciated that throughout the present description of embodiments, discussions utilizing terms such as “displaying”, “performing”, “capturing”, “comparing”, “determining”, “accessing”, “presenting”, or the like, often refer to the actions and processes of a computer system, or similar electronic computing device such as a tablet computer. The computer system or similar electronic computing device manipulates and transforms data represented as physical (electronic) quantities within the computer system's registers and memories into other data similarly represented as physical quantities within the computer system memories or registers or other such information storage, transmission, or display devices. Embodiments of the present technology are also well suited to the use of other computer systems such as, for example, mobile communication devices including tablet computers.

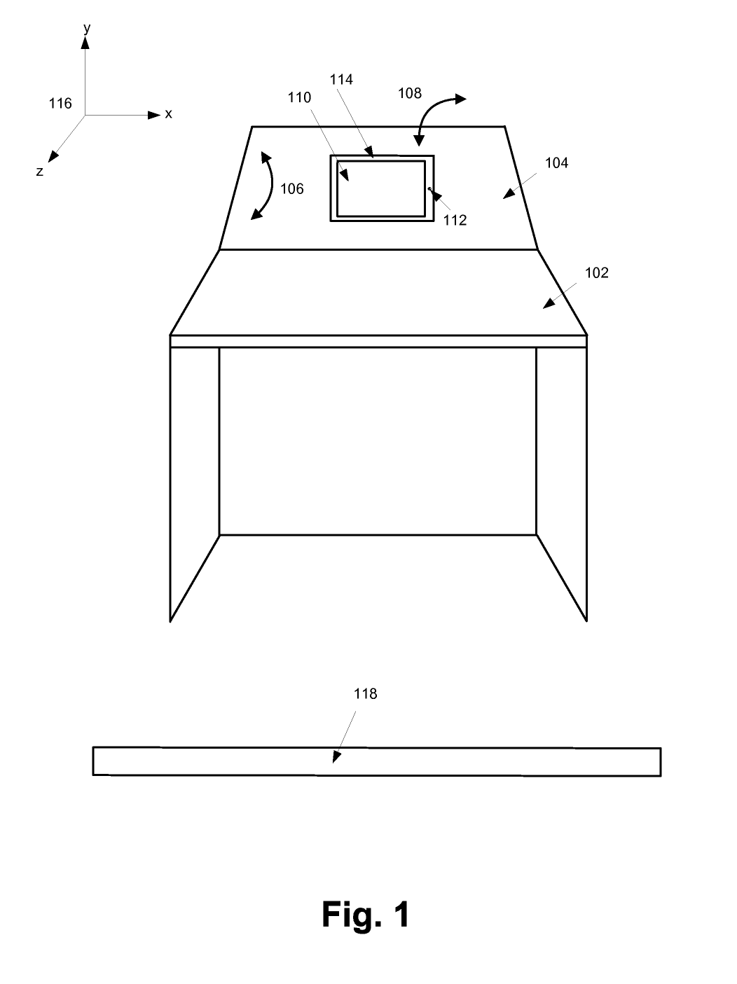

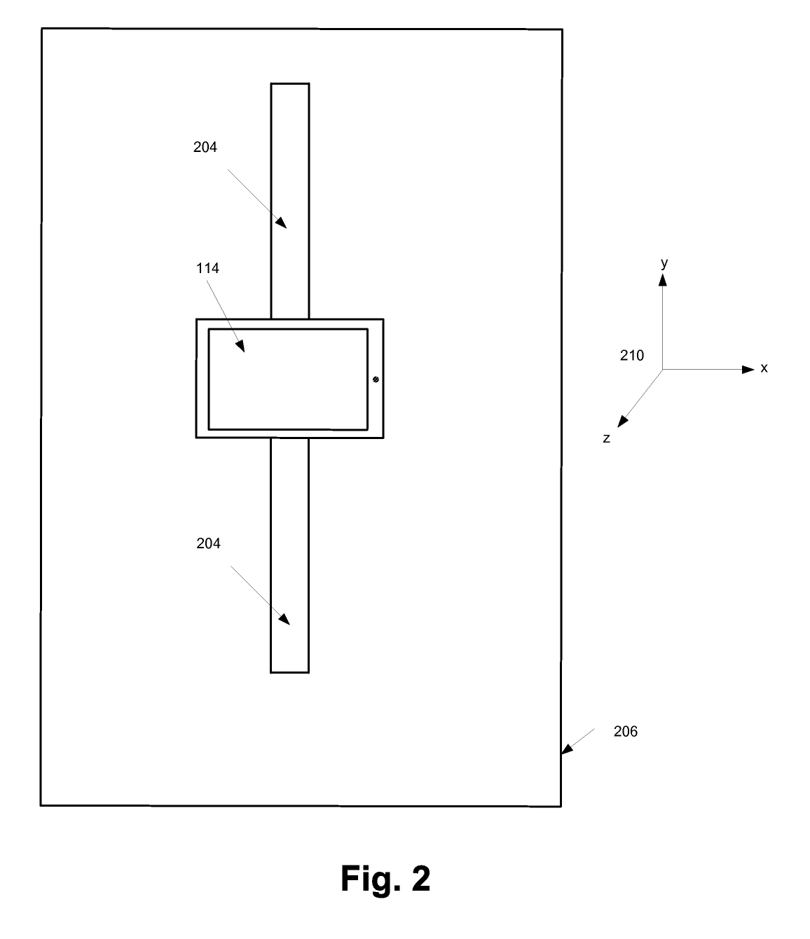

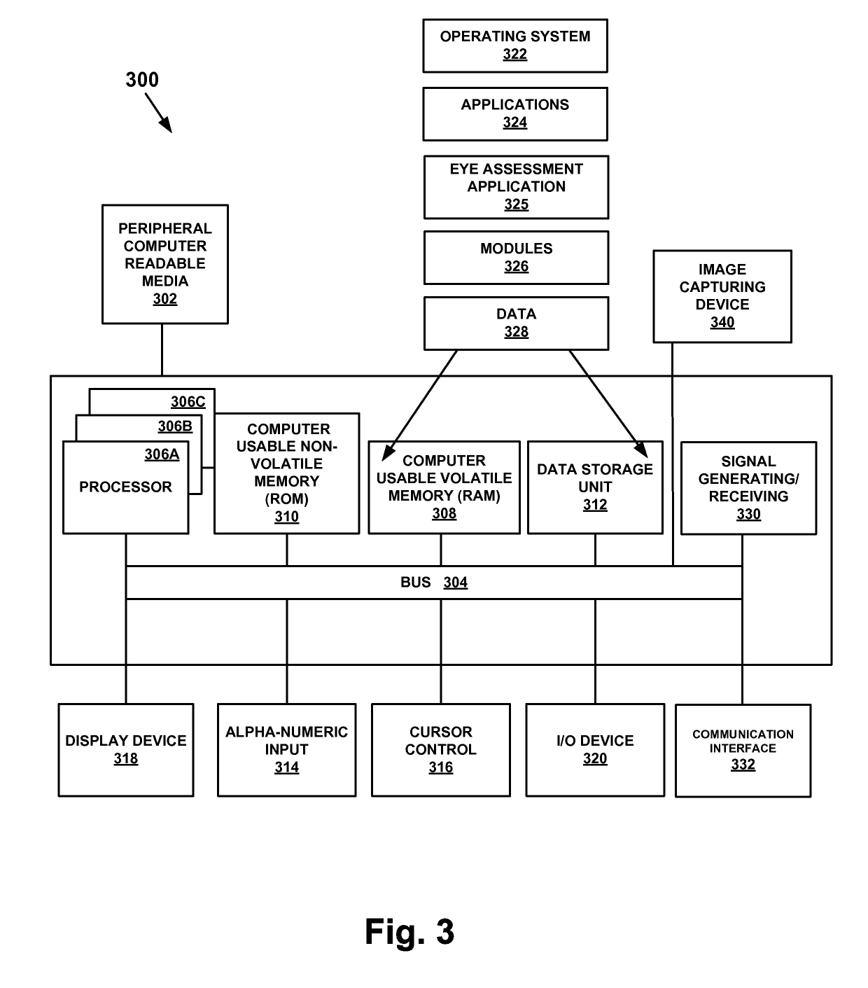

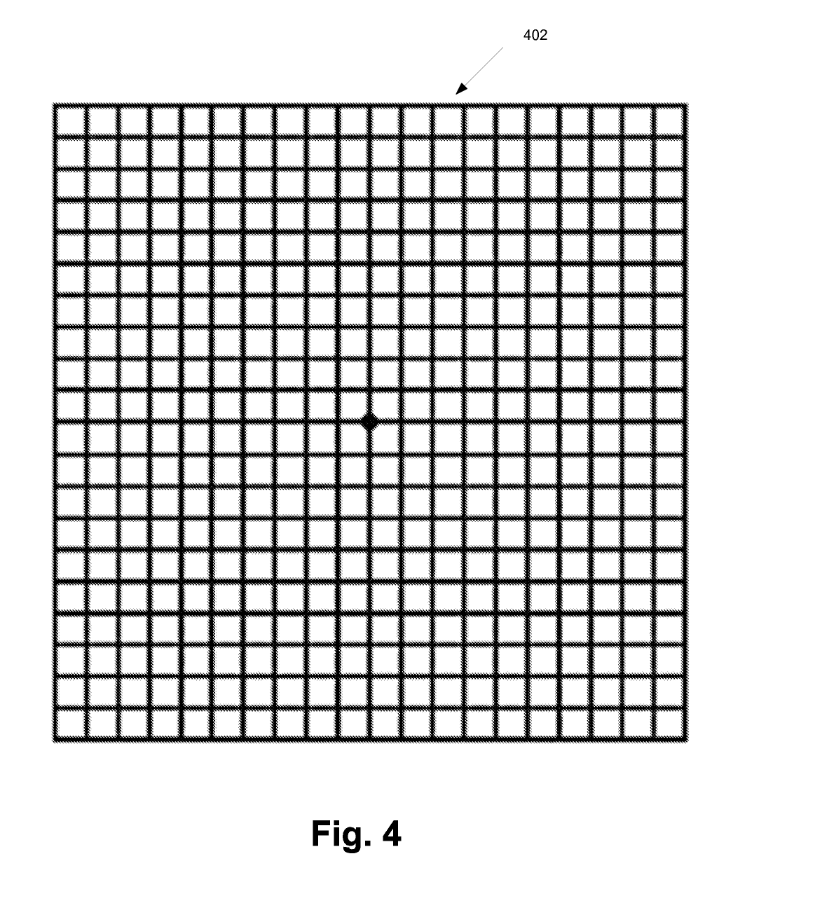

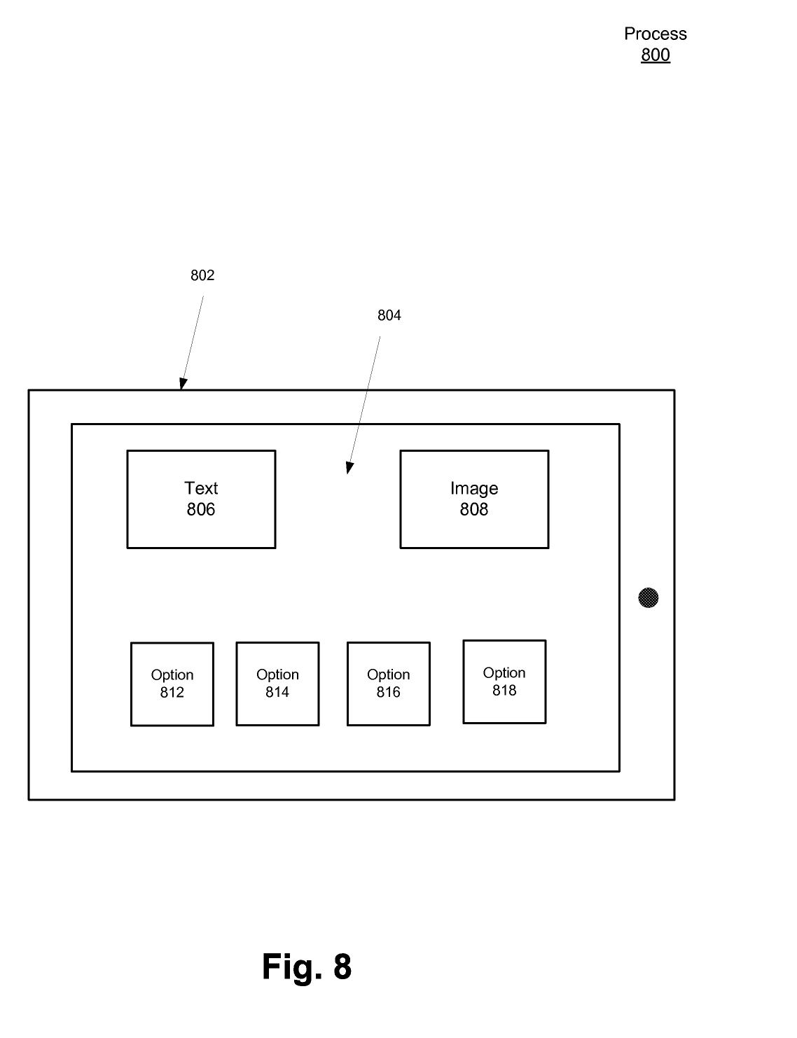

The discussion below begins with a general overview of embodiments. The discussion follows with a description of a station for performing an assessment of vision-related health conditions (See FIGS. 1 and 2), in accordance with an embodiment. Following, an example computer system upon which embodiments of the present technology may be implemented (See FIG. 3) is described, in accordance with embodiments. Following, various images for performing an assessment of vision-related health conditions (See FIGS. 4-7) are described, in accordance with embodiments. A flowchart of a method for performing an assessment of vision-related health conditions (See FIG. 8) is then shown, in accordance with embodiments.

### Overview

Embodiments described herein are for performing an assessment of vision-related health conditions. The embodiments describe assessments related to or associated with vision or eye related diseases. In one embodiment, the assessments are for assessing a person's visual acuity; however, the assessment may also pertain to other vision-related health impairments. It should be appreciated that the assessment may be described as a test, screening, or evaluation. In one embodiment, the assessments described herein do not refer to a test administered by a licensed healthcare professional such as an ophthalmologist or optometrist but rather providing information and options to a person to assist the person in determining if a healthcare professional is required.

The present technology employs a high resolution display associated with a computer system and an application to provide an interactive experience to a person or user where images are displayed on the high resolution display and the user is then queried regarding the images. The user's answers or responses to the queries are then employed to assess, evaluate, or determine health-related conditions pertaining to the user. The user is then presented with options for connecting with a licensed healthcare professional, a marketing representative, or both. The options for connecting are based on the assessment or evaluation for the particular user.

In one embodiment, the high resolution display associated with the computer system is a tablet computer system such as an iPad. The tablet computer system may be installed or embedded at a station such as a kiosk where the tablet computer system is the only electronic device required and used for the assessment.

The term “station” as used herein may refer to a station that is permanent, stationary, or fixed, or may refer to a station that is temporary, mobile, or portable. For example, the station may be a fixture and permanently attached to a structure such as a wall or floor where the table computer system is embedded in a structure made of a material such as wood or plastic. Conversely, the station may be portable and may simply be a tablet computer system that is set on a table top or may be station that is installed in a vehicle that moves the station from one location to another and a potential user enters the vehicle to use the station. In one embodiment, the station is a kiosk that is self-contained and may be placed in an environment such as a retail setting. For example, the kiosk may be placed in a pharmacy.

### Interactive Platform for Health Assessment

With reference now to FIG. 1, a block diagram of a station for performing an assessment of vision-related health conditions for a user. FIG. 1 comprises structure 102 which may be made of any number of materials including metal, wood, plastic, etc. Structure 102 also comprises component 104. Component 104 is for the mounting of or embedding of tablet computer 114. In one embodiment, tablet computer 114 is embedded in component 104 such that tablet computer 114 is not easily removable from structure 102 but display 110 of tablet computer 114 is visible. Tablet computer 114 may be embedded in component 104 such that no physical buttons, switches, or ports of tablet computer 114 are visible or accessible from the front of structure 102. This prevents undesired tampering of tablet computer 114 from members of the public. In one embodiment, only display 110 and camera 112 of tablet computer 114 are visible. In one embodiment, structure 102 and/or component 104 comprise holes or ports for audio from a speaker of tablet computer 114 to pass through to a user of structure 102.

Tablet computer 114 may be networked to other computers via a local network or the Internet. In one embodiment, tablet computer 114 has hardware components that allow tablet computer 114 to connect to the Internet via a cellular network. In one embodiment, tablet computer 114 makes use of a wired connection to connect to a network. In one embodiment, tablet computer 114 makes use of a local wireless network to connect to the Internet.

Coordinates 116 indicate the three dimensional nature of the station in the x, y and z directions. In one embodiment, component 104 can be moved relative to base portion of structure 102 in the z and y direction as is indicated by arrow 106. Thus the angle of display 110 can be changed relative to a user's view of display 110. This ability to change the angle may be useful patients of different height. In one embodiment, tablet computer 114 is able to change positions relative to component 104 but is still embedded in component 104 and is not easily removed. Arrow 108 demonstrates how tablet computer 114 can be moved in the x and y direction.

In one embodiment, structure 102 has no electronics or computer components but is only a structure for housing tablet computer 114. In such an embodiment, all of the assessments and evaluations are performed only by tablet computer 114. In one embodiment, the only electronics that are a component of or housed by structure 102, other than tablet computer 114, is a power source for tablet computer 114.

Structure 102 in combination with tablet computer 114 may be referred to as a station. A user of the station may stand or sit while interacting with tablet computer 114. Tablet computer 114, as part of the instructions for the assessment, may instruct the user to stand or sit. The instructions may also direct the user as to how far away the user's eyes should be from display 110. In one embodiment, indicator 118 is an indication on the floor near structure 102 that indicates a designated place the user should stand while performing the interactions with tablet computer 114 for the assessment. Indicator 118 may be considered a component of the station. In one embodiment, a user enters their height into tablet computer 114 and that information is then used to direct the user how far away to place their eyes from display 110. In one embodiment, the visual acuity test is generally performed with a 40 cm separation between the user's face and the display screen on the tablet computer.

In one embodiment, structure 102 comprises text or images on the surfaces of structure 102. The text or images may have information regarding the interactive health assessment for which tablet computer 114 is employed or may be advertisements. The advertisements may be paid for by a third party and may advertise products related to vision and eye healthcare. In one embodiment, structure 102 comprises lights, sounds, or other devices to attract potential users.

In an embodiment, tablet computer 114 comprises a high-resolution screen, an audio output, a microphone, a camera 112, memory and a processor, and is operated by a software program to perform a variety assessments and/or evaluations of a user's visual acuity. It should be appreciated that the present technology is described herein as being carried out using a tablet computer; however, any type of computer system with a high resolution display may be used. Tablet computers and computer systems in general are described in more detail with reference to FIG. 3.

In one embodiment, camera 112 of tablet computer 114 may be used to take a close-up picture of a user's eye or eyes. The resolution available on currently available tablet computers is sufficient to provide an image suitable for analysis by a health care professional, to diagnose a variety of possible ailments or diseases associated with the cornea or the white part of the eyeball. In one embodiment, camera 112 captures video of the user's eye. The video may be analyzed by tablet computer 114 to calculate a blink rate of the user. The blink rate may then further be used to make an assessment about the user's health condition.

In one embodiment, the image of a user's eyes may be forwarded to a health care professional via an Internet link. Contact with such a healthcare professional can be made by selecting from a menu of available resources listed on the screen after the tests are performed as will be seen below.

With reference now to FIG. 2, a block diagram of a station for performing an assessment of vision-related health conditions for a user. FIG. 2 comprises track 204. In one embodiment, tablet computer 114 is attached, connected to, or coupled with track 204 and tablet computer 114 is able to easily move along track 204 to adjust the height or position of tablet computer 114. Different heights of users may require a different position of tablet computer 114. In one embodiment, a user may enter their height into tablet computer 114 after which the user is instructed as to which height to move the tablet computer 114 along track 204. Track 204 may comprise measurement marks indicating to the user what height the tablet computer 114 is at when moved to a particular measurement indicator. In one embodiment, a user may be able to move the position of tablet computer 114 along track 204 without being able to remove tablet computer 114 off of track 204. Thus tablet computer 114 may not be easily stolen. Track 204 may be mounted to wall 206. Tablet computer 114 coupled with track 204 mounted to wall 206 may be described as a station and may be located in a public venue such as a pharmacy. Coordinates 210 show the three dimensional nature of the tablet computer 114 and the station. As depicted, tablet computer 114 may change positions along the y axis. However, more elaborate or complex tracks may be employed to move the tablet computer 114 in other dimensions than only the y axis.

In an embodiment, track 204 may be a vertically oriented rod attached to a portion of a wall or the station, thus providing an up-down adjustment along the vertical extent of the rod, and may include a swivel mount to move the tablet computer left or right, or change the elevation angle to permit proper alignment of the screen with the user's face.

With reference to FIG. 8, a block diagram of a tablet computer with an interface in accordance with embodiments of the present technology. It should be appreciated that tablet computer 802 has all the features and abilities described for tablet computer 114 of FIG. 1. Interface 804 depicts an interface that may be displayed to the user for an interactive experience that is used for the assessment or evaluation of health conditions related to the vision of a user. Interface 804 used the technique of displaying an image, such as the letter E, posing a question to the user, and then receiving the user's response creates an interactive experience that is used for the assessment or evaluation. It should be appreciated that interactive experience may employ various techniques and media for interacting with the user. The media may include text, queries, images, audible sounds or words, or any combination thereof. The user may be requested to enter data via hardware buttons, a virtual keyboard on a touchscreen, selecting options on a touchscreen, speaking into a microphone, or posing for a picture.

Text 806, of interface 804, may provide information to the user regarding the assessment or may be a query posed to the user for the assessment. A query may refer to an image such as image 808. The image may be directly used to perform an assessment for visual acuity or may simply be an image to enhance the usability or personalize the interactive experience. In one embodiment, text 806 and image 808 are third party advertisements displayed to the user. Options 812, 814, 816, and 818 may refer to options that a user may select in response to a query or may be options presented to the user and be based on the evaluation or assessment. For example, the options may be to contact a licensed healthcare professional, a marketing representative, or both.

In one embodiment, results or information of the assessment may not be displayed on display 110 because display 110 may be in a public area where other people may view the results. The results may be private and confidential and therefore should not be displayed in public out of respect and also for legal reasons. Therefore, options 812, 814, 816, and 818 may be options for the user to retrieve their results. One of the options may be to have the results emailed or sent electronically to the user. One of the options may be to display a Quick Response Code system (QR™) code to the user. The user may then use a device such as a smart phone to capture the QR code and then use their device to extract data from the QR code which may lead the device to take further actions such as access a website. The QR code may also be employed to access content from third parties associated with the station such as sponsors, partners, or advertisers.

It should be appreciated that that interface 804 may be more than one portion of text, images, options, etc. The interactive experience may take a user through several different displays in interface 804. Each different display in interface 804 may be based on a user's input or response to a previous query. In other words, two different users may not see the same series of images, text and queries in interface 804 and may not be given the same options, or they may be displayed in different order. In an embodiment, the interactive experience may be guided by the user. For example, additional tests may be administered, depending on the desire of the user. Various applications may operate on the tablet computer to provide evaluations for assessing health conditions such as macular degeneration, dry eye, a dryness level of the eye, various allergies, level of ocular fatigue, presence of trachoma, presence of cataracts, color blindness, and level of vision acuity. Examples of images used for the assessment are found in FIGS. 4-7.

In one embodiment, before a user is able to use a station and tablet computer 114 for the assessment, the user may be required to enter user information into tablet computer 114. The user information may be to login to an existing using account, create a new account, create a single use account, or other options. The user information may identify a user by name or may simply require the user to enter contact information such as an email address, phone number, or phone number that receives text messages. The user information may or may not be password protected. The user may enter data into tablet computer 114 via a virtual keyboard on the touchscreen of tablet computer 114 or other means. In one embodiment, if a user has an existing account with previous assessments, the previous assessments may be used in conjunction with current evaluation and testing to create a new assessment.

In one embodiment, during the interactive experience the user may be asked to rate a symptom on a numerical or other scale. Examples of symptoms that may be questions are eyes that are sensitive to light, eyes that feel gritty, painful or sore eyes, blurred vision, poor vision, swollen or puffy eyes, watery eyes, red eyes, itchy or burning eyes, or dry eyes. The user may be asked to rank on a scale of 1-6 or 1-4 for the symptoms regarding a period of time such as the past week. The same symptoms may be questioned differently such as ranking how often the user suffered from the symptom and then questioned how much the user was troubled by the symptom.

With reference to FIG. 4, a block diagram of an image used for performing an assessment of vision-related health conditions for a user in accordance with embodiments of the present technology. In one embodiment, tablet computer 114 displays image 402 in an interface. Image 402 is an example vision test grid or Amsler grid. The grid is a tool that eye doctors use to detect vision problems resulting from damage to the macula (the central part of the retina) or the optic nerve. Tablet computer 114 may display image 402 and then give directions to the user. The directions may be to cover one eye, wear reading glasses if necessary, then look at the dot in the center of the grid of image 402. The user may then be queried whether the user sees waviness, sees the corners of the grid, sees dark spots, missing lines, etc. Both eyes may be tested. The input received from the user is then employed to create an assessment of the user's eye health in regards to macular degeneration or other eye diseases.

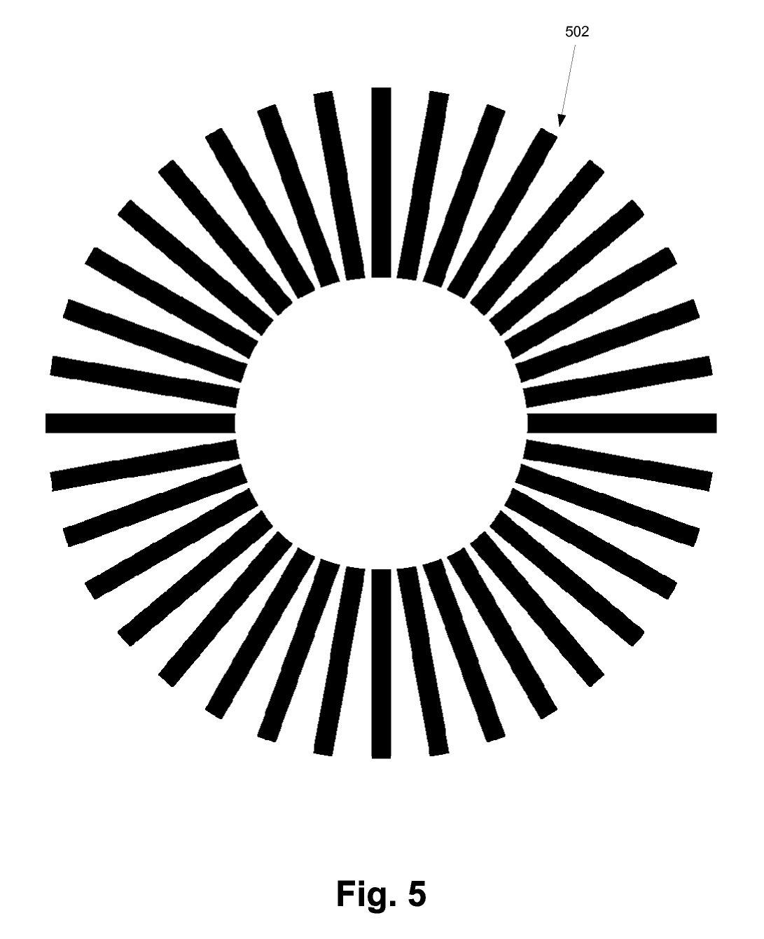

With reference to FIG. 5, a block diagram of an image used for performing an assessment of vision-related health conditions for a user in accordance with embodiments of the present technology. In one embodiment, tablet computer 114 displays image 502 in an interface. Image 502 is an example of an image used for evaluating astigmatism. Tablet computer 114 may display image 502 and then give directions to the user. The directions may be to cover, then look at image 502. The user may then be queried whether some of the lines in image 502 appear grayer and some blacker. Both eyes may be tested. The input received from the user is then employed to create an assessment of the user's eye health in regards to astigmatism or other eye diseases.

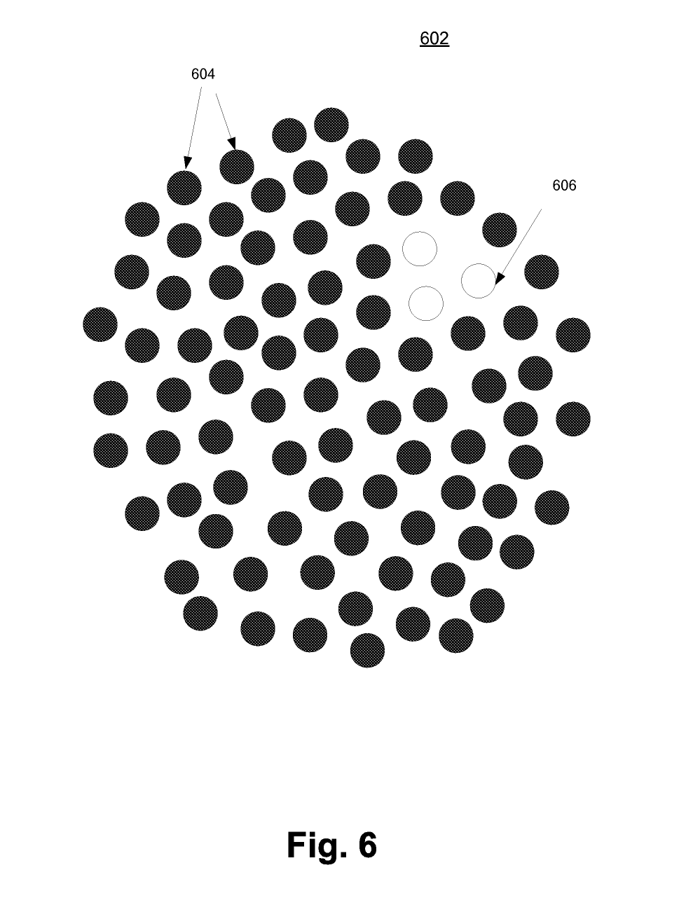

With reference to FIG. 6, a block diagram of an image used for performing an assessment of vision-related health conditions for a user in accordance with embodiments of the present technology. In one embodiment, tablet computer 114 displays image 602 in an interface. Image 602 is an example of an image used for evaluating color blindness. Image 602 may display circles or dots such as dots 604 and 606. It should be appreciated that dots 604 and 606 in image 602 are all uniform in size; however, the dots need not be the same size. Dots 604 and 606 and depicted as being different in color than one another, dots 604 are black and dots 606 are white. In practice, the dots may be different colors than black and white. For example, dots 604 may be green and 606 may be red. The colors green and red are often used to test for color blindness, but other colors may be used as well. The dots are arraigned such that one color of dots forms a pattern within the other dots. Image 602 depicts the white dots 606 forming a triangle within the black dots 604. The pattern may more typically be a numeral or a letter that is easily recognized. Tablet computer 114 may display image 602 and then give directions to the user. The directions may be to look at image 602. The user may then be queried whether any pattern, letters, or numerals are visible to the user. The input received from the user is then employed to create an assessment of the user's eye health in regards to color blindness or other eye diseases.

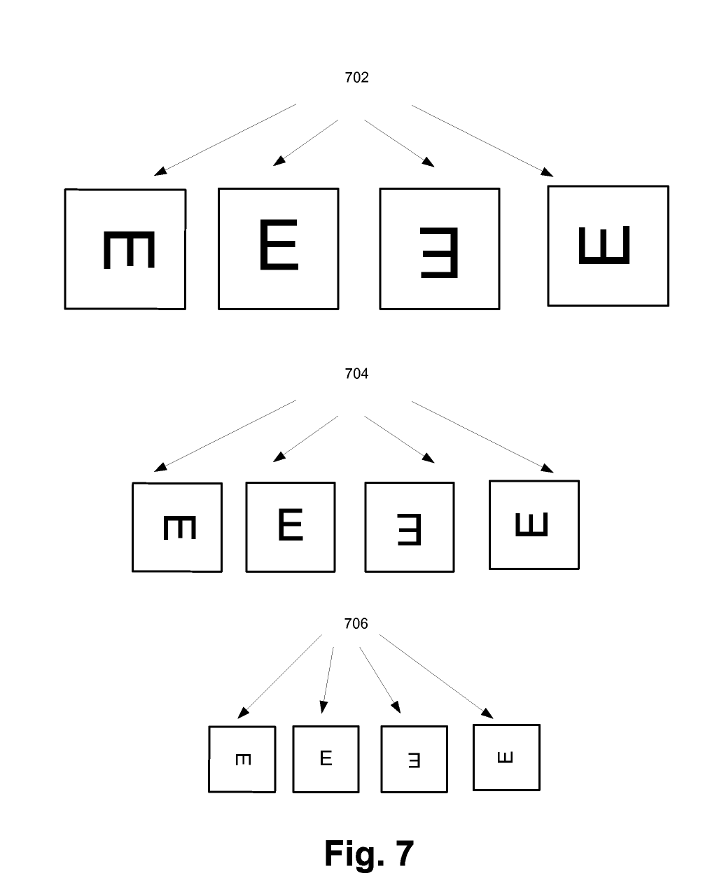

With reference to FIG. 7, a block diagram of an image used for performing an assessment of vision-related health conditions for a user in accordance with embodiments of the present technology. In an embodiment, tablet computer 114 operates a program that displays a capital letter [usually a C or an E] in any of four orientations [regular, reversed left-right, rotated clockwise, rotated counter-clockwise] in ever-decreasing sizes. This is demonstrated in FIG. 7 with a capital letter E at 702, 704, and 706. The user must select a replica shown below the test letter with the displayed orientation as proof the user can actually see the shape and orientation of the letter. If display 110 is a touchscreen then the user may enter the selection via display 110 by pressing an option in the interface. In one embodiment, the high-resolution screen on a tablet computer like the Retina Display on the 9.7 in iPad has sufficient resolution, meaning small pixel size, to permit a decent representation of a capital letter like the E. FIG. 7 may be employed to create an assessment of the user's eye health in regards to visual acuity or other eye diseases.

In making an assessment about a user's visual acuity, it may be necessary to display images on tablet computer 114 at specific sizes using a specific number of pixels on the display. In one embodiment, to show a capital E, it should be at least 5 pixels in height, and 3-4 in width. In an embodiment using a screen with 2048-by-1536 resolution at 264 pixels per inch (ppi), means that 5 pixels are 5/264=0.01893 inch or about 19 thousandths. In one embodiment, this is the smallest Capital E that can be shown on such a display. In one embodiment, the letters are displayed at 12 pixels in height.

In one embodiment, after the interactive experience has gathered sufficient data for an assessment, the interface may display a variety of professional services that are available to the user via the interactive experience. For example, the user may consult with a licensed healthcare professional directly via video chat. The video chat may use a service such as Skype or FaceTime and employ tablet computer 114 to carry out the video chat. The data such as a QR code may also be used for the user to gain access to the video chat with the licensed healthcare professional. The present technology may provide the licensed healthcare professional with the assessment created or generated by tablet computer 114. Thus the licensed healthcare professional may have a starting point to intelligently interact with the user. Alternately, local vision and/or healthcare professionals' contact information is also presented. Such vision care professionals may include optometrists, ophthalmologists, medical doctor, allergists, contact lens or glasses manufacturers, or the like.

In one embodiment, the present technology may connect the user to a marketing representative based on the assessment. For example, it may be assessed that the user is experiencing dry eyes or allergies. The user may then be presented with an option to connect with a marketing representative. The marketing representative may be sponsored by a third party manufacturer who is a trusted content provider and provides products or information to consumers that are related to the user's health condition such as dry eyes. The marketing representative may be a representative of a manufacturer, vendor, seller, distributor, third party administrator (TPA), other product/service provider, etc. and may be licensed or unlicensed. Two examples of trusted content providers are Allergan or Latisse. The user may be offered an incentive to connect with the marketing representative such as a coupon or discount that is available if the connection is made or made within a certain time frame. The marketing representative may or may not have the user's assessment made available to them. A user may elect to make the assessment available to either a licensed healthcare professional or a marketing representative.

It should be appreciated that the option to connect with a licensed healthcare professional or a marketing representative may take place using one or more of several communications means such as texting, instant messaging, a phone call, a voice call via Internet means, video chatting, a link to a website, email, etc. The connection may take place using the tablet computer at the station or may take place via the user's device. The user's device may receive information for the communication using several techniques. The user's device may be sent a text or an email or a QR code may be displayed via tablet computer 114, which is then captured by the user's device. The connection to a licensed healthcare professional or marketing representative may be made immediately after the assessment or may be made at a later time. The connection may also be referred to as an electronically facilitated communication. In one embodiment, the user may provide contact information to tablet computer 114 and request that a licensed healthcare professional and/or a marketing representative contact the user.

### Definition of Visual Acuity

Acuteness of vision; it is indicated by a fraction in which the numerator is a number expressing the distance in feet at which the patient sees a line of type on the chart (usually 20 feet), and the denominator a number expressing the distance in feet at which the normal eye would see the smallest letters which the patient sees at the distance at which he/she is; thus, if at 20 feet he/she sees only the letters, which the normal eye would see at 50 feet, the formula of his/her vision acuity will be 20/50. This is the definition only referring to DVA (Distance Visual Acuity).

### Kiosk Integration Embodiment

In one embodiment, the station for eye health assessment is an embedded screen integrated into a kiosk. For example, at various pharmacies, a health care kiosk is provided where a health test, such as a blood pressure test can be performed. In one embodiment, the eye health system of the present technology is integrated into a kiosk such as this where one or more health-related tests can be performed. In one embodiment, a mounting bezel is provided that securely couples the computer system or display device to the kiosk. In one embodiment, an existing kiosk can be retrofitted with a standalone tablet type device for performing eye heath evaluations. In one embodiment, a locking bezel is used provide a secure coupling between the kiosk and the electronic tablet-type device.

### Computer System

With reference now to FIG. 3, portions of the technology for providing a communication composed of computer-readable and computer-executable instructions that reside, for example, in non-transitory computer-usable storage media of a computer system. That is, FIG. 3 illustrates one example of a type of computer that can be used to implement embodiments of the present technology. FIG. 3 represents a system or components that may be used in conjunction with aspects of the present technology. In one embodiment, some or all of the components of FIGS. 1, 2, 4, 5, 6, 7 and 8 may be combined with some or all of the components of FIG. 6 to practice the present technology.

With reference now to FIG. 3, portions of the technology for providing a communication composed of computer-readable and computer-executable instructions that reside, for example, in computer-usable media of a computer system. That is, FIG. 3 illustrates one example of a type of computer that can be used to implement embodiments of the present technology.

FIG. 3 illustrates an example computer system 300 used in accordance with embodiments of the present technology. It is appreciated that system 300 of FIG. 3 is an example only and that the present technology can operate on or within a number of different computer systems including general purpose networked computer systems, embedded computer systems, routers, switches, server devices, user devices, various intermediate devices/artifacts, stand-alone computer systems, mobile phones, personal data assistants, televisions and the like. As shown in FIG. 3, computer system 300 of FIG. 3 is well adapted to having peripheral computer readable media 302 such as, for example, a floppy disk, a compact disc, and the like coupled thereto. In one embodiment, system 300 is tablet computer such as tablet computer 114 of FIG. 1.

System 300 of FIG. 3 includes an address/data bus 304 for communicating information, and a processor 306A coupled to bus 304 for processing information and instructions. As depicted in FIG. 3, system 300 is also well suited to a multi-processor environment in which a plurality of processors 306A, 306B, and 306C are present. Conversely, system 300 is also well suited to having a single processor such as, for example, processor 306A. Processors 306A, 306B, and 306C may be any of various types of microprocessors. System 300 also includes data storage features such as a computer usable volatile memory 308, e.g. random access memory (RAM), coupled to bus 304 for storing information and instructions for processors 306A, 306B, and 306C.

System 300 also includes computer usable non-volatile memory 310, e.g. read only memory (ROM), coupled to bus 304 for storing static information and instructions for processors 306A, 306B, and 306C. Also present in system 300 is a data storage unit 312 (e.g., a magnetic or optical disk and disk drive) coupled to bus 304 for storing information and instructions. System 300 also includes an optional alpha-numeric input device 314 including alphanumeric and function keys coupled to bus 304 for communicating information and command selections to processor 306A or processors 306A, 306B, and 306C. System 300 also includes an optional cursor control device 316 coupled to bus 304 for communicating user input information and command selections to processor 306A or processors 306A, 306B, and 306C. System 300 of the present embodiment also includes an optional display device 318 coupled to bus 304 for displaying information.

Referring still to FIG. 3, optional display device 318 of FIG. 3 may be a liquid crystal device, cathode ray tube, plasma display device or other display device suitable for creating graphic images and alpha-numeric characters recognizable to a user. Optional cursor control device 316 allows the computer user to dynamically signal the movement of a visible symbol (cursor) on a display screen of display device 318. Many implementations of cursor control device 316 are known in the art including a trackball, mouse, touch pad, joystick or special keys on alpha-numeric input device 314 capable of signaling movement of a given direction or manner of displacement. Alternatively, it will be appreciated that a cursor can be directed and/or activated via input from alpha-numeric input device 314 using special keys and key sequence commands.

System 300 is also well suited to having a cursor directed by other means such as, for example, voice commands. System 300 also includes an I/O device 320 for coupling system 300 with external entities. For example, in one embodiment, I/O device 320 is a modem for enabling wired or wireless communications between system 300 and an external network such as, but not limited to, the Internet. A more detailed discussion of the present technology is found below. System 300 also includes image capture device 340 which may be a camera such as camera 112 of FIG. 1.

Referring still to FIG. 3, various other components are depicted for system 300. Specifically, when present, an operating system 322, applications 324, modules 326, and data 328 are shown as typically residing in one or some combination of computer usable volatile memory 308, e.g. random access memory (RAM), and data storage unit 312. However, it is appreciated that in some embodiments, operating system 322 may be stored in other locations such as on a network or on a flash drive; and that further, operating system 322 may be accessed from a remote location via, for example, a coupling to the Internet. In one embodiment, the present technology, for example, is stored as an application 324 or module 326 in memory locations within RAM 308 and memory areas within data storage unit 312. The present technology may be applied to one or more elements of described system 300. Eye assessment application 325 is the application described above for providing the interactive experience to the user and for displaying images, querying a user, receiving input from the user, and performing an assessment via the hardware of system 300. Eye assessment application 325 can also provide options to the user to connect with a licensed healthcare professional or a marketing representative.

System 300 also includes one or more signal generating and receiving device(s) 330 coupled with bus 304 for enabling system 300 to interface with other electronic devices and computer systems. Signal generating and receiving device(s) 330 of the present embodiment may include wired serial adaptors, modems, and network adaptors, wireless modems, and wireless network adaptors, and other such communication technology. The signal generating and receiving device(s) 330 may work in conjunction with one or more communication interface(s) 332 for coupling information to and/or from system 300. Communication interface 332 may include a serial port, parallel port, Universal Serial Bus (USB), Ethernet port, antenna, or other input/output interface. Communication interface 332 may physically, electrically, optically, or wirelessly (e.g. via radio frequency) couple system 300 with another device, such as a cellular telephone, radio, or computer system.

The computing system 300 is only one example of a suitable computing environment and is not intended to suggest any limitation as to the scope of use or functionality of the present technology. Neither should the computing environment 300 be interpreted as having any dependency or requirement relating to any one or combination of components illustrated in the example computing system 300.

The present technology may be described in the general context of computer-executable instructions, such as program modules, being executed by a computer. Generally, program modules include routines, programs, objects, components, data structures, etc., that perform particular tasks or implement particular abstract data types. The present technology may also be practiced in distributed computing environments where tasks are performed by remote processing devices that are linked through a communications network. In a distributed computing environment, program modules may be located in both local and remote computer-storage media including memory-storage devices.

### Operations

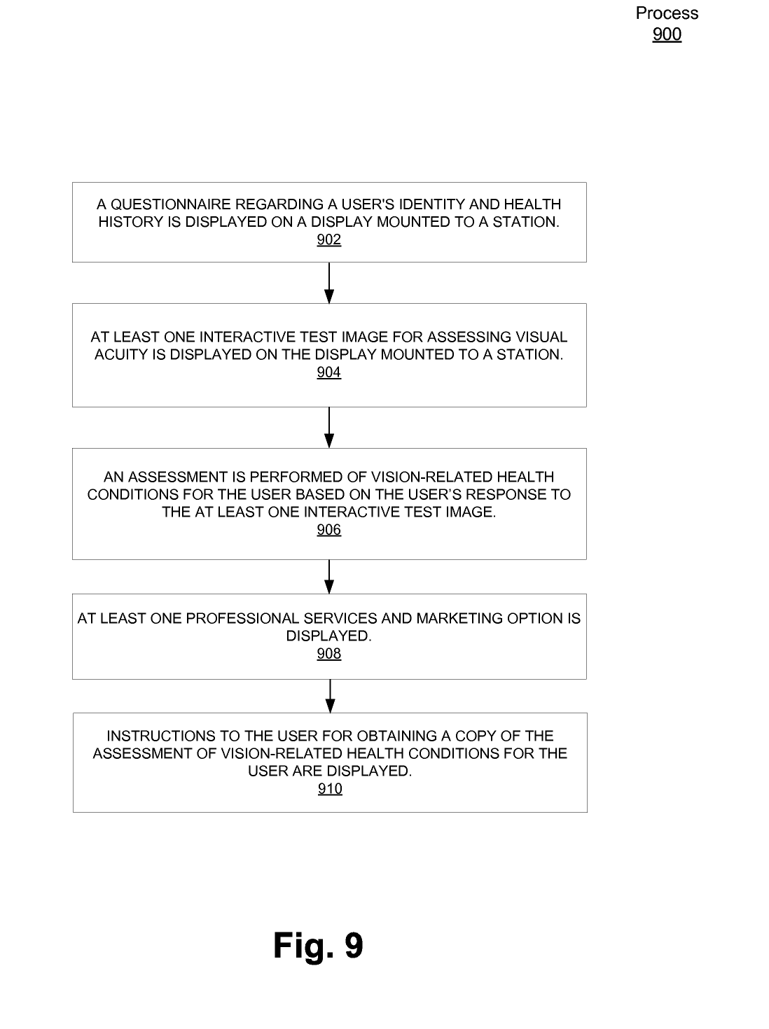

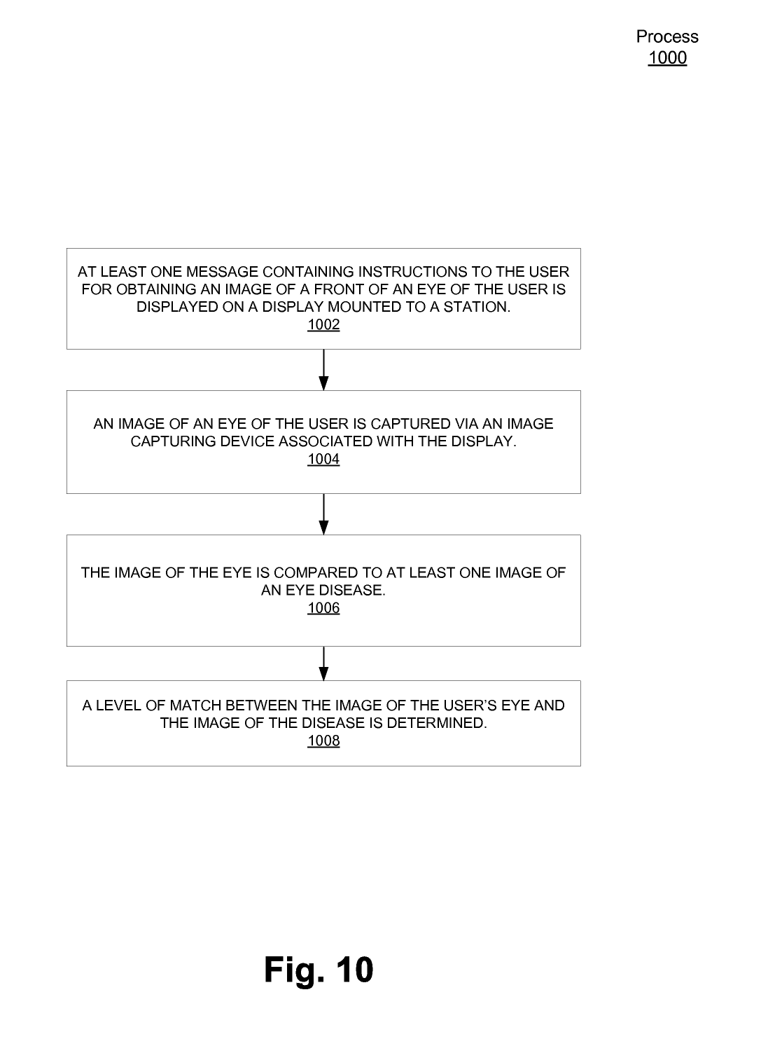

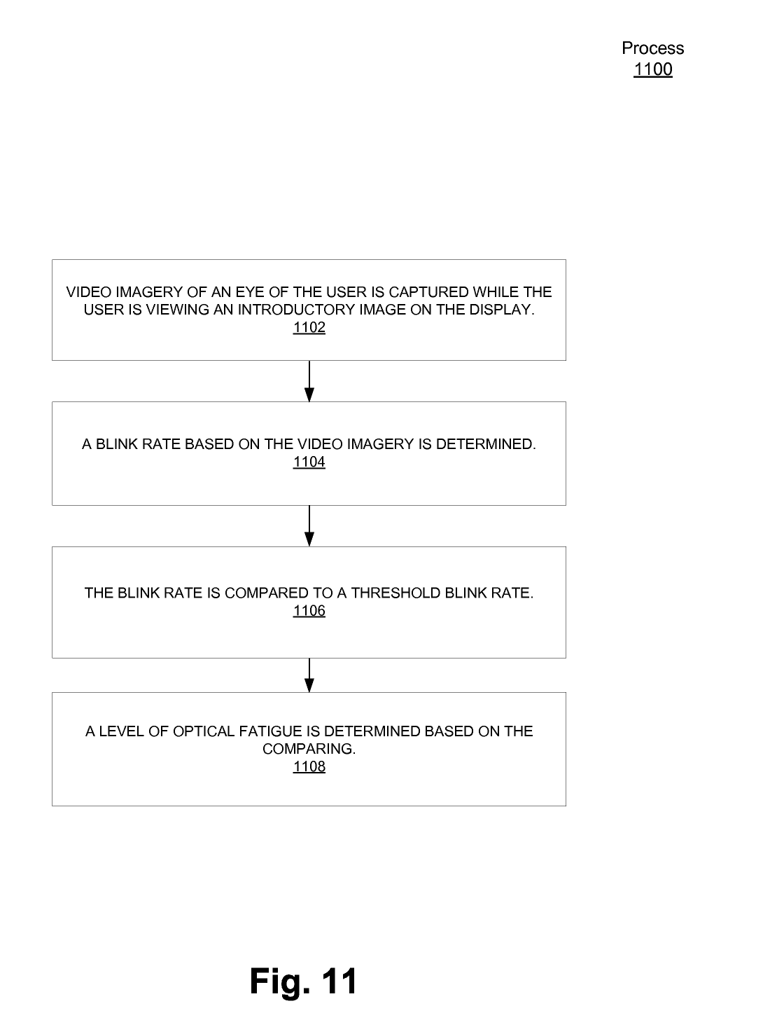

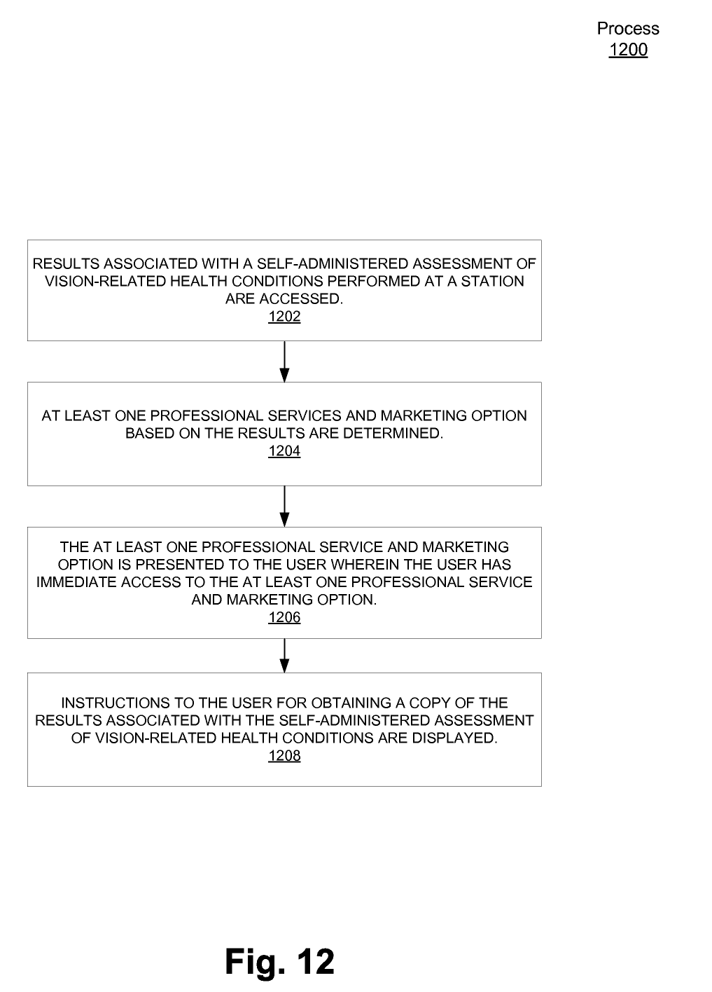

With reference to FIGS. 9, 10, 11, and 12, processes 900, 1000, 1100, and 1200 are for performing an assessment of vision-related health conditions for a user. In one embodiment, processes 900, 1000, 1100, and 1200 are computer implemented methods that are carried out by processors and electrical components under the control of computer usable and computer executable instructions. The computer usable and computer executable instructions reside, for example, in data storage features such as computer usable volatile and non-volatile memory. However, the computer usable and computer executable instructions may reside in any type of non-transitory computer usable storage medium. In one embodiment, processes 900, 1000, 1100, and 1200 are performed by the components of FIG. 1, 2, 3, 4, 5, 6, 7, or 8. In one embodiment, the methods may reside in a computer usable storage medium having instructions embodied therein that when executed cause a computer system to perform the method.

It should be appreciated that each of the processes comprises a number of steps. The steps may or may not be performed in the order described and a given step may or may not be performed in a given embodiment of the process. Process 900 may be performed independently or may be performed in conjunction with processes 1000, 1100 and/or 1200.

At 902, a questionnaire regarding a user's identity and health history is displayed on a display mounted to a station. This may be performed at a station using a computer such as tablet computer 114 of FIG. 1. In one embodiment, 902 identifies the user or receiving contact information for the user. In one embodiment, the user is not required to enter all requested information and may not enter health history information. Examples of information requested of the user may be birthdate, does user wear prescription glasses, contact lenses, reading glasses or sunglasses, when was the users last eye exam, has the user been diagnosed with an eye related disease.

At 904, at least one interactive test image for assessing visual acuity is displayed on the display mounted to a station. For example, the images in FIGS. 4, 5, 6 and 7 may be used.

At 906, an assessment is performed of vision-related health conditions for the user based on the user's response to the at least one interactive test image. This can be accomplished via tablet computer 114.

At 908, at least one professional services option is displayed. The professional services option may be to connect with a licensed healthcare professional or marketing representative.

At 910, instructions to the user for obtaining a copy of the assessment of vision-related health conditions for the user are displayed. This could be a link, an email, or a QR code, or other option.

Process 1000 may be performed may be performed in conjunction with processes 900, 1100 and/or 1200 and is for performing the assessment is for assessing optical fatigue.

At 1002, at least one message containing instructions to the user for obtaining an image of a front of an eye of the user is displayed on a display mounted to a station. Tablet computer 114 of FIG. 1 may be used.

At 1004, an image of an eye of the user is captured via an image capturing device associated with the display. Camera 112 of tablet computer 114 of FIG. 1 may be used.

At 1006, the image of the eye is compared to at least one image of an eye disease. The at least one image of an eye diseased may be stored locally on tablet computer 114 or may be accessed at a remote database via a network such as the Internet.

At 1008, a level of match between the image of the user's eye and the image of the disease is determined.

Process 1100 may be performed independently or may be performed in conjunction with processes 900, 1000 and/or 1200 and is for performing the assessment is for assessing optical fatigue.

At 1102, video imagery of an eye of the user is captured while the user is viewing an introductory image on the display. Camera 112 of tablet computer 114 of FIG. 1 may be used.

At 1104, a blink rate based on the video imagery is determined. Tablet computer 114 may be used to analyze the video imagery.

At 1106, the blink rate is compared to a threshold blink rate. The threshold blink rate may be stored on tablet computer 114.

At 1108, a level of optical fatigue is determined based on the comparing.

Process 1200 may be performed independently or may be performed in conjunction with processes 900, 1000 and/or 1100 and is for presenting healthcare options to a user of a medical testing device.

At 1202, results associated with a self-administered assessment of vision-related health conditions performed at a station are accessed. The results may be accessed by tablet computer 114 of FIG. 1 but may not be displayed due to privacy concerns. The results may be accessed via tablet computer 114, or a server computer associated with tablet computer 114, or other device not associated with the station. The device accessing the results may retrieve the results from the station or from a database associated with the station.

At 1204, at least one professional services option based on the results are determined. The step may occur at the station or computer associated with the station and then the options are sent to a computer not associate with the station.

At 1206, the at least one professional service and marketing option is presented to the user wherein the user has immediate access to the at least one professional service and marketing option.

At 1208, instructions to the user for obtaining a copy of the results associated with the self-administered assessment of vision-related health conditions are displayed.

Although the subject matter is described in a language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

1. A computer-usable storage medium having instructions embodied therein that when executed cause a computer system to perform a method for performing an assessment of vision-related health conditions for a user, said method comprising:
displaying on a display mounted to a station, a questionnaire regarding a user's identity and health history;
displaying on said display mounted to said station, at least one interactive test image for assessing visual acuity;
performing an assessment of vision-related health conditions for said user based on said user's response to said at least one interactive test image; and
displaying at least one professional services and marketing option.

2. The computer-usable storage medium as recited in claim 1, further comprising:
capturing an image of an eye of said user via an image capturing device associated with said display.

3. The computer-usable storage medium as recited in claim 2, further comprising:
comparing said image of said eye to at least one image of an eye disease; and
determining a level of match between said image of said user's eye and said image of said disease.

4. The computer-usable storage medium as recited in claim 1, further comprising: displaying on said display mounted to said station, instructions to said user for obtaining a copy of said assessment of vision-related health conditions for said user.

5. The computer-usable storage medium as recited in claim 1, wherein said at least one professional service is an option for said user to make a connection with a service provider and said option is selected from the group of options consisting of: an electronically facilitated discussion with a licensed healthcare professional; sending an email to a licensed healthcare professional; a video chat session with a licensed healthcare professional; an electronically facilitated discussion with a marketing representative; sending an email to a marketing representative; and a video chat session with a marketing representative.

6. The computer-usable storage medium as recited in claim 1, wherein said performing an assessment of vision-related health conditions is selected from the group of assessments consisting of: an assessment for evaluating dry eye, assessment for evaluating at least one type of allergy, assessment for evaluating a level of eye dryness, assessment for a level of ocular fatigue,
assessment for a presence of trachoma, assessment for a presence of cataracts, and assessment for a level of vision acuity.

7. The computer-usable storage medium as recited in claim 1, further comprising:
displaying on said display mounted to said station, at least one message containing instructions to said user for obtaining an image of a front of an eye of said user.

8. The computer-usable storage medium as recited in claim 1, wherein said performing said assessment is for assessing optical fatigue, further comprising:
capturing video imagery of an eye of said user while said user is viewing an introductory image on said display;
determining a blink rate based on said video imagery;
comparing said blink rate to a threshold blink rate; and
determining a level of optical fatigue based on said comparing.

9. The computer-usable storage medium as recited in claim 1, wherein said displaying said at least one professional services option comprises displaying a quick response (QR) code for said user to capture with a device, said QR code then provides said device with said at least one professional service option.

10. A computer-usable storage medium having instructions embodied therein that when executed cause a computer system to perform a method for presenting healthcare options to a user of a medical testing device, said method comprising:
accessing results associated with a self-administered assessment of vision-related health conditions performed at a station;
determining at least one professional services option based on said results; and
presenting at said station said at least one professional service option to said user wherein said user has immediate access to said at least one professional service option.

11. The computer-usable storage medium as recited in claim 10, further comprising:
comparing an image of an eye of said user to at least one image of an eye disease; and
determining a level of match between said image of said user's eye and said image of said disease.

12. The computer-usable storage medium as recited in claim 10, further comprising: displaying on said display mounted to said station, instructions to said user for obtaining a copy of said results associated with said self-administered assessment of vision-related health conditions.

13. The computer-usable storage medium as recited in claim 10, wherein said at least one professional service is an option for said user to make a connection with a service provider and said option is selected from the group of options consisting of: an electronically facilitated discussion with a licensed healthcare professional; sending an email to a licensed healthcare professional; a video chat session with a licensed healthcare professional; an electronically facilitated discussion with a marketing representative; sending an email to a marketing representative; and a video chat session with a marketing representative.

14. The computer-usable storage medium as recited in claim 10, wherein said self-administered assessment of vision-related health conditions is selected from the group of assessments consisting of: an assessment for evaluating dry eye, assessment for evaluating at least one type of allergy, assessment for evaluating a level of eye dryness, assessment for a level of ocular fatigue, assessment for a presence of trachoma, assessment for a presence of cataracts, and assessment for a level of vision acuity.

15. A system coupled with a station for testing a user for performing an assessment of vision-related health conditions for a user, comprising:
a display coupled with a station for displaying, questionnaire regarding a user's identity and health history;
an interactive image generator configured to display on said display, at least one interactive test image for assessing visual acuity;
a service option database for storing a plurality of professional services options; and
an evaluator for performing an assessment of vision-related health conditions for said user based on said user's response to said at least one interactive test image, and for selecting and providing at least one professional service option from said plurality of professional services options, wherein said selecting is based said assessment.

16. The system as recited in claim 15, further comprising:
an adjustable bezel mount for coupling said display to said station and configured to enable adjustment of said display with respect to a size said user.

17. The system of claim 15 further comprising:
an image capturing device associated with said display for capturing an image of an eye of said user.

18. The system of claim 17 further comprising:
a memory associated with said display for storing said image of said eye.

19. The system of claim 17 further comprising:
a comparison engine configured for comparing said image of said eye to at least one image of an eye disease.

20. The system of claim 19 further comprising:
a match determiner for determining a level of match between said image of said user's eye and said image of said eye disease.
